(19) United States
(12) Patent Application Publication
(10) Pub. No.: US 2005/0137807 A1
(43) Pub. Date: Jun. 23, 2005

(54) Method of visualization of the ADME properties of chemical substances

(75) Inventors: Jorg Lippert, Leverkusen (DE); Michael Sevestre, Leverkusen (DE); Walter Schmitt, Neuss (DE); Stefan Willmann, Dusseldorf (DE)

Correspondence Address: Norris, McLaughlin & Marcus P.A., 875 Third Avenue -18th Floor, New York, NY, 10022, US

(73) Assignee: Bayer Technology Services GmbH, Leverkusen (DE)

(21) Appl. No.: 10/971,458

(22) Filed: Oct. 22, 2004

(30) Foreign Application Priority Data
Oct. 29, 2003 (DE) 10350525.3

(51) Int. Cl. A01N 61/00; G06F 19/00; G01N 33/48; G01N 33/50; A01N 25/00

(52) U.S. Cl. 702/19; 514/1; 504/116.1

(57) ABSTRACT

A method is described for selecting chemical compounds and visualizing their ADME properties using an indication-specific target profile. In one embodiment, the method comprises determining and/or selecting molecular properties of one or more compounds in a computer database. This information is used to generate one or more ADME maps describing the compounds' behaviour in a biophysical model. An indication-specific target profile of the desired ADME properties is defined and compared with the compounds' actual ADME profile or map to make an optimized selection of compounds.

The invention relates to a computer system and a method for the visualisation of ADME properties for a multiplicity of chemical substances, and subsequent selection as well as automatic filtering of the substances with the aid of a predetermined requirement profile. This invention is based on an earlier development (DE 101 60 270 A1) and, in relation to it, represents an extension and improvement which greatly simplifies the data evaluation and interpretation.

A goal in all fields of chemical research is to synthesize substances which fulfil a particular predetermined requirement profile. Medical active agents, for example, must be capable of reaching the place in the body where they are intended to act (“target”) in order to exhibiting the intended biochemical effect there (for example inhibition of an enzyme, etc.).

In order to obtain early information about the likely physical, biological, biochemical, pharmacological or other relevant properties of a substance which has not yet been fully characterized experimentally (and possibly not yet synthesized), structure-property relationships are compiled according to the prior art. Such structure-property relationships are established in many fields of application, such as for the classification of potential active agents in medicinal chemistry or agrochemistry, for assessment of the toxicity of chemical substances, for the early estimation of polymer or catalyst properties, etc.

In the field of ADME properties (A=absorption, D=distribution, M=metabolism, E=excretion), which is particularly relevant to pharmaceutical active-agent research, substance properties such as lipophilicity, solubility, permeability across artificial membranes or cell layers, molecular weight and numbers of particular structural features, for example hydrogen donors and acceptors, are usually taken into account. The assessment of the substances then generally involves compliance with particular limits, which are usually obtained from empirical values, expert knowledge or from the statistical distribution of the properties of commercially available products. One extensively used known guideline, which was derived in this way, is Lipinski's “Rule of Five” for describing orally administered active agents (C. A. Lipinski et al., Adv. Drug Del. Rev. 23, pp. 3-25 (1997)). A crucial disadvantage of such a method (as described in DE 101 60 270 A1), is that they will consider rigid limits for individual properties which are only indirectly relevant. The ADME properties which are actually important, however, generally depend simultaneously on a plurality of these quantities. The tolerable limit for an individual quantity is therefore not in fact a constant, rather it changes its value as a function of the values of other relevant quantities. An improved method, which takes such dependencies into account by the incorporation of complex biophysical models, is described in DE 101 60 270 A1.

On the basis of the technique described in DE 101 60 270 A1, the present invention relates to an improved method which, through calculation of the ADME properties for a multiplicity of chemical substances, allows visualization of the properties in the form of so-called ADME maps and subsequent graphical selection and automatic filtering of particularly suitable active-agent candidates with the aid of a predetermined requirement profile, and to a corresponding computer program and method.

Visualization of the ADME properties by means of such ADME maps is advantageous compared with a representation of the ADME property in table form (as described in DE 101 60 270 A1), since it compares and contrasts all the substances of the substance library at a glance and therefore allows very straightforward and rapid assessment of the substances in relation to the ADME property.

Methods for the visualisation of complex data structures are known per se, and are commercially available in the form of software tools (for example Origin from the OriginLab Corporation or Spotfire). Such pure visualisation tools, however, are configured without any application-specific “intelligence”, i.e. they represent data as it stands but do not per se carry out any interpretation of the information or selection of candidates.

The direct linking of a biophysical model with a visualisation tool as described in the present application is novel, as is the combination with application-specific, indication-dependent requirement profiles which relate directly to the ADME properties (and not, as is customary according to the prior art, to the molecular structure of properties). Besides manual selection of particularly suitable active-agent candidates, therefore, automatic filtering and substance assessment may also be carried out. This may either be applied to substance libraries with hundreds of thousands of individual substances, as are nowadays customary in industrial pharmaceutical research, or in the scope of active-agent research projects to assist decision and making project control.

The invention relates to a method for the visualization of ADME properties and for the selection of chemical substances and structures with the aid of an indication-specific target profile, with the following steps:
a) determining or selecting and subsequently entering molecular properties of a multiplicity of substances or chemical structures into a computer system,
b) setting up one or more ADME maps by means of one or more biophysical models of possible expressions of substance properties for molecules in a selected molecular weight range,
c) linking the chemical structures in a) with the biophysical models in b) and optionally representing the structures as data points in the ADME maps from b) (“mapping”),
d) defining an indication-specific target profile in the ADME property space,
e) classifying the structures with respect to the target profile, for example up to a molecular weight of 1000, and selection with the aid of the classification.

The molecular properties according to a) preferably involve a selection from the following properties:
lipophilicity, binding constant to plasma proteins, molecular weight, molecular volume, water solubility, solubility in intestinal fluid, permeability coefficient across a biological membrane, fraction unbound in plasma, kinetic constants of a metabolic process, kinetic constants of an active transport process.

It is preferable to use, as the biophysical model, one or more respectively selected from the list:
physiology-based pharmacokinetic model for mammals
physiology-based pharmacokinetic model for insects
physiology-based pharmacokinetic model for plants.

The ADME properties preferably involve a selection of the following:
For the case of a model for mammals:
fraction unbound in plasma, organ/blood distribution coefficient, organ/plasma distribution coefficient, distribution volume, terminal half-life in blood, plasma or an organ, intestinal permeability, absorbed fraction of a dose of the substance following oral application, maximum concentration in the blood, plasma or an organ.

In the case of a model for plants:
characteristic for the rate of absorption into the leaf following a spray application, characteristic for the rate of distribution in the plant following leaf application (phloem mobility), characteristic for the rate of distribution in the plant following root application (xylem mobility).

In the case of a model for insects:
characteristic for the rate of absorption into an insect through the gut following oral application, characteristic for the rate of absorption into an insect through the cuticle following topical application.

In a preferred method, the target profile is obtained from empirical values, expert knowledge and/or the statistical distribution of relevant ADME properties for known substances.

The classification is particularly preferably carried out using truth values which represent the fulfilment of an individual requirement of an ADME the property.

As an alternative, the classification is particularly preferably performed by combining a plurality of truth values, which represent the fulfilment of an individual requirement, by means of Boolean algebra.

In another preferred variant of the method, the classification is performed by means of an index value, which quantifies the deviation from a target value.

In another preferred version of the method, the classification is performed by means of a weighted average of a plurality of index values, which quantify the deviation from a target value.

Another preferred variant of the method is characterized in that the classification is performed by means of a probability value, which indicates the probability rank in relation to an empirical distribution function obtained from known substances for an ADME property.

The input of the substance properties may be performed by importing values from a substance database or by using substance information obtained from experiments, which is available in particular as a file.

The selection and filtering may be performed by the user of the computer system using graphical selection, or may be carried out automatically by the computer system using predetermined requirement profiles.

Examples of complex biophysical models are physiology-based pharmacokinetic (PBPK) models. Such models are known according to the prior art. A PBPK model for mammals has been mathematically described in detail, for example by Kawai et al. (R. KAWAI, M. LEMAIRE, J.-L. STEIMER, A. BRUELISAUER, W. NIEDERBERGER, M. ROWLAND, “Physiologically Based Pharmacokinetic Study on a Cyclosporin Derivative, SDZ IMM 125”, J Pharmacokin. Biopharm. 22, 327-365 (1994)). A PBPK model for lepidoptera larvae has been described by Greenwood et al. (R. GREENWOOD, M. G. FORD, E. A. PEACE, D. W. SALT: “The kinetics of Insecticide Action. Part IV: The in vivo Distribution of Pyrethroid Insecticides during Insect Poisoning” Pestic. Sci. 30, 97-121 (1990)), an example of a PBPK model for plants is the model by Satchivi et al. (Satchivi N. M., Stoller, E. W., Wax L. M., Briskin D. P., A nonlinear dynamic simulation model for xenobiotic transport and whole plant allocation following foliar application Parts I and II. Pest. Biochem. and Physiol. 2000; 68: 67-95).

The basic principle is represented in FIG. 1. The starting point is a library or database of chemical structures (11), which contains molecular properties (12) for a multiplicity of structures. These molecular properties may either have been found experimentally beforehand, or may have been determined with the aid of structure-based prediction methods which are known per se, such as QSAR or neural networks.

In a first step, the “ADME map” (14) is set up for the ADME property of interest. An ADME map is a two-dimensional representation, in particular encoded with false colours or contours, of the ADME property as a function of two or more molecular substance properties due to the structure, on which this ADME property depends. The calculation is preferably carried out—as described in DE 101 60 270 A1—with the aid of biophysical models (13).

The so-called “mapping” is carried out in a second step, i.e. the substances contained in the substance library are represented as data points in this ADME map (15). The position of any given substance in this ADME map is determined by its respective molecular structure properties. Optionally, additional information may also be represented within an ADME map, for example further molecular structure properties or ADME properties derived from them, the synthesis date, the name of the synthesis chemist etc., for example encoded by colour, symbol or size modulation of the data points. In this way, for example, it is readily possible to reconstruct the historical development of an active-agent research project.

The selection of the substances takes place in the third step. A target profile which the substances to be selected should ideally have in relation to the ADME property (or alternatively which they should on no account have) is defined for the selection (16). In the scope of the invention, the term “indication-specific target profile” is intended to mean selected criteria and values which specify an intended ADME property. The target profile for the ADME property is application-specific. The target profile usually defines a subregion of the ADME map. As such, it may also be highlighted optically, for example by means of bounding lines or by variation of the representation parameters (shade of colour, saturation, etc.) on the colour ADME map. Comparing the position of any given substance on the ADME map with the target profile makes it possible to assess the substances (17).

Steps one to three may be carried out similarly for further relevant ADME properties, so that a substance assessment can be carried out overall on the basis of a plurality of ADME properties.

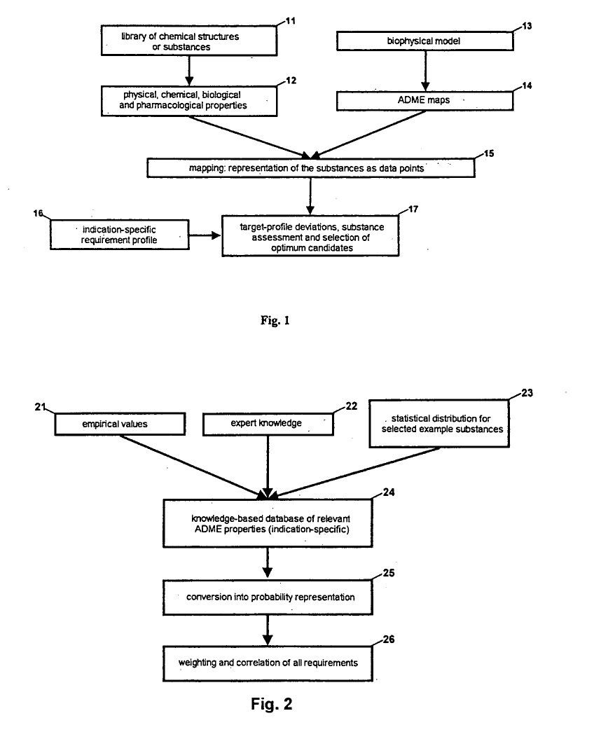

A preferred method for the definition of a target profile is represented in FIG. 2. First, a knowledge-based database is prepared about advantageous (and/or particularly disadvantageous) ADME properties (24). Sources for this knowledge-based database are, for example, empirical values (21), expert knowledge (22) and/or similarly to the procedure of C. A. Lipinski et al. [C. A. Lipinski et al., Adv. Drug Del. Rev. 23, 3-25 (1997)]—even the statistical distribution of relevant ADME properties for commercially available products (23) (N.B.: but specifically for the ADME property and not just for the molecular structure property!). Suitable sources for such analyses are, for example, databases such as the World Drug Index, the Red List, the Pesticide Manual, the PhysProp database, NCI databases, Medline, etc. The requirements placed on the ADME properties for active agents are generally indication-specific. From this knowledge database (25), a statistical distribution function for each individual ADME property can then be derived which indicates the probability that a particular ADME property will have a particular value. These probability representations may be employed individually for the classification, or combined to form an individual value (index) by weighted correlation of the individual probability representations (26).

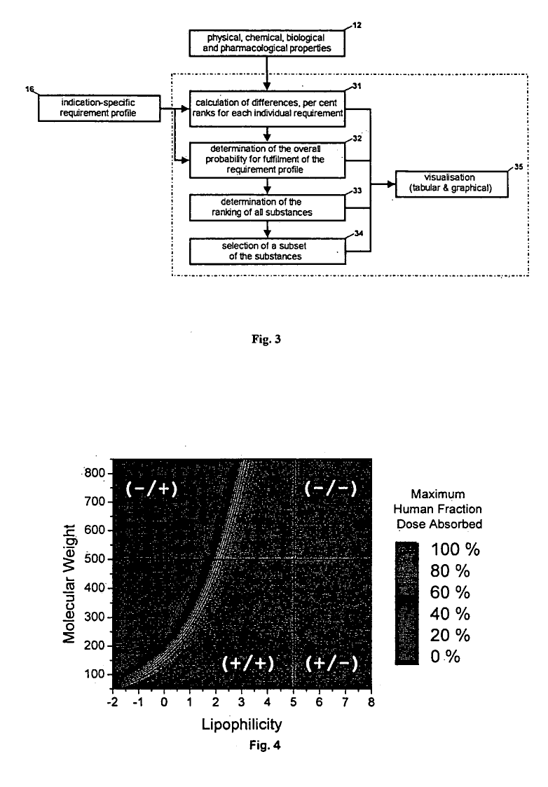

A preferred method for the subsequent assessment of the substances is represented in FIG. 3. Each data point is then studied on each ADME map to see whether it belongs to the target profile space. This may, for example, be done in the scope of a qualitative classification in which a check is made to see whether a data point lies inside or outside the target region (yes/no analysis), or a quantitative classification through generation of an index value (31). In the latter variant, absolute or relative weightings are calculated for each individual requirement (for example based on the distance of a data point from the boundary line of the target profile, or as a probability value which is derived from the empirical distributions for known commercially available substances). The weighted sum of the individual classifiers may be calculated in order to form a overall index value (32). This overall index value determines the ranking of the substances (33). The result, which represents a subset of the original substances (34), may be output as a table or in the form of graphs (35).

### Examples

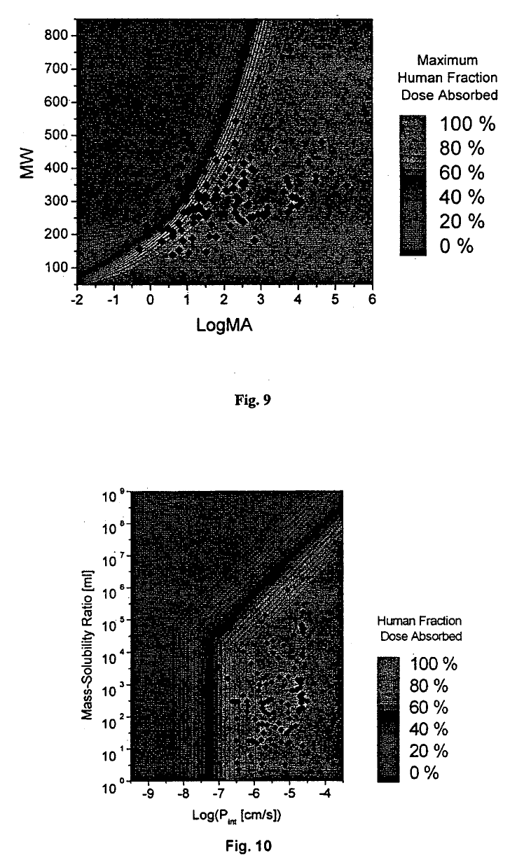

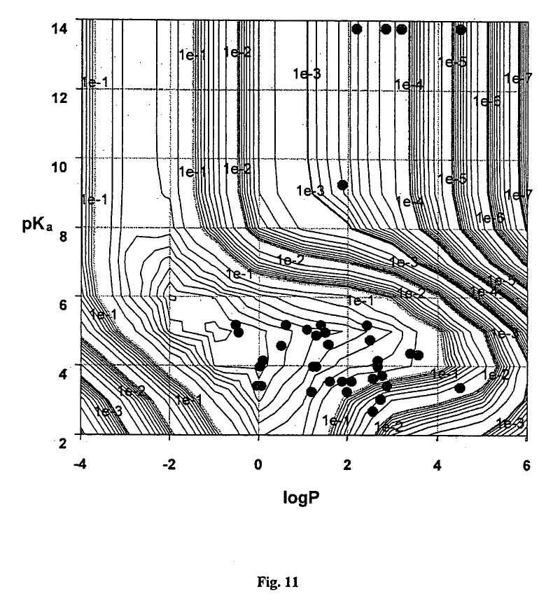

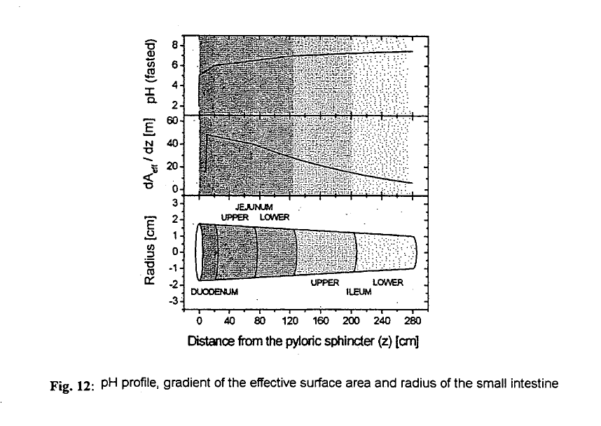

The subsequent examples of the present invention are based on the following biophysical model: The ADME maps in FIGS. 4 and 9 for the maximum absorbed fraction of an orally administered dose and FIG. 10 for the fraction dose absorbed, on the basis of a continuous model for gastrointestinal flux and absorption of an oral dose. This model combines physiological influencing factors, such as geometrical dimensions, pH profile and effective surface area of the gastrointestinal tract, with a physiological flux profile described via an intestinal transit function (Tₛᵢ(z,t)) and two substance-dependent parameters: the intestinal permeability (Pᵢₙₜ) and the intestinal solubility (Sᵢₙₜ). The relevant physiological parameters are represented summarily in FIG. 12.

The transit profile defines the fraction of an orally administered dose at a position z in the small intestine (z=0 defines the pylorus) at a time t (after oral administration of the substance). Based on an experimental data record by Sawamoto et al. (T. Sawamoto, S. Haruta, Y. Kurosaki, K. Higaki and T. Kimura. Prediction of the Plasma Concentration Profiles of Orally Administered Drugs in Rats on the basis of Gastrointestinal Transit Kinetics and Absorbability, J. Pharm. Pharmacol. 49: 450-457 (1997)), it was approximated by a Gaussian function with time-variable centroid zₒ(t) and width σ(t):

$${T}_{S I} ( z , t ) = \frac{1 - exp \{ - t / {τ}_{GE} \}}{\sqrt{2 π} σ ( t )} exp \{ - \frac{{( z - {z}_{o} ( t ) )}^{2}}{2 {σ}^{2} ( t )} \} \qquad ( 1 )$$

Here, τ_{GE} denotes the time constant for release of the substance from the stomach into the intestine, which was assumed to be 30 min in the model. The time-variable parameters z₀(t) and σ(t) are approximated by an exponential function and a ninth-order polynomial

$$\begin{matrix}{z}_{o} ( t ) = α + {β ( t - {t}_{0} )}^{n} & ⋀ & σ ( t ) = {\sum }_{k = 0}^{9} {γ}_{k} {t}^{k}\end{matrix} \qquad ( 2 )$$

with the coefficients:

| Model parameter | Value |
|---|---|
| α | −6.1 |
| B | 10.43 |
| t₀ | 0.07 |
| n | 0.081 |
| γ₀ | 0.32191 |
| γ₁ | 2.86798 |
| γ₂ | −6.89234 |
| γ₃ | 8.01795 |
| γ₄ | −5.19735 |
| γ₅ | 2.04239 |
| γ₆ | −0.50334 |
| γ₇ | 0.07631 |
| γ₈ | −0.0065 |
| γ₉ | 0.000237493 |

The concentration of the substance at the position z in the intestinal lumen at time t can be calculated from this as follows:

$${C}_{lumen} ( z , t ) = \frac{DOSE BW ( 1 - {f}_{abs} ( t ) )}{π {r}^{2} ( z ) {L}_{SI}} {T}_{SI} ( z , t ) \qquad ( 3 )$$

Here, DOSE denotes the administered dose, BW stands for the body weight, L_{S1} is the total length of the intestine (=280 cm), f_{abs}(t) is the fraction already absorbed at time t. The solubility may limit the amount absorbed, since the substance precipitates in the gastrointestinal tract if luminal concentrations locally occur which exceed the value of the solubility (Sᵢₙₜ). This case is taken into account by a threshold condition, which always limits the luminal concentration to the value of the intestinal solubility:

$${C}_{lumen} = \{ \begin{matrix}{C}_{lumen} , & if {C}_{lumen} \leq {S}_{int} \\ {S}_{int} , & if {C}_{lumen} > {S}_{int}\end{matrix} \qquad ( 4 )$$

Overall, the amount of substance amount of substance which is absorbed across the intestinal membrane into the portal vein in the region [z. . . . z+dz] in the time interval [t . . . t+dt] is therefore obtained as:

$$\frac{{ⅆ}^{2} {M}_{pv} ( z , t )}{ⅆ z ⅆ t} = {P}_{int} {C}_{lumen} ( z , t ) \frac{ⅆ {A}_{eff} ( z )}{ⅆ z} \qquad ( 5 )$$

Numerical integration of this differential equation with respect to positions provides the absorption profile of the substance as a function of time, and integration with respect to time provides the amount absorbed overall in a segment of the gastrointestinal tract. The fraction absorbed overall (Fraction Dose Absorbed) is given by:

$${F}_{abs} = {\int }_{t = 0}^{∞} {\int }_{z = 0}^{{L}_{SI}} \frac{{ⅆ}^{2} {M}_{pv} ( z , t )}{ⅆ z ⅆ t} ⅆ z ⅆ t / ( DOSE BW ) \qquad ( 6 )$$

With the assumption that the solubility does not have any limiting influence (i.e. Cₗᵤₘₑₙ<Sᵢₙₜ is satisfied at all times for any position), the maximum absorbed fraction of an orally administered dose which is represented in FIGS. 4 and 9 is obtained. FIG. 10 shows the general case with solubility limitation.

The intestinal permeability is therefore the only quantity which determines the maximum absorbed fraction of an orally administered dose. Between this quantity and the physicochemical substance parameters of lipophilicity (MA) and molecular weight (MW), there is a biophysical relationship which is given by the following equation:

$${P}_{int} ( MW , MA ) = A \frac{{MW}^{- α - β} MA}{{MW}^{- α} + B M {W}^{- β} MA} + C \frac{{MW}^{- γ}}{{D}^{- γ} + {MW}^{- γ}} [ cm \text{/} s ] \qquad ( 7 )$$

The parameters A, B, C, D, α, β and γ have the values:

| A | B | C | D | α | β | γ |
|---|---|---|---|---|---|---|
| 7440 | 1.0 × 10⁷ | 2.5 × 10⁻⁷ | 202 | 0.60 | 4.395 | 16 |

The first example shows an ADME map for the maximum absorbed fraction of an orally administered dose in humans, which was calculated according to the method described above with the aid of a physiology-based pharmacokinetic model. In addition, two selection criteria known according to the prior art for oral active agents, which belong to Lipinski's “Rule of Five”, are also shown as lines (lipophilicity <5 and molecular weight <500).

According to the Lipinski rules, for example, active agents are unsuitable for passive absorption following oral administration if they have a lipophilicity >5 and a molecular weight >500 (identified by (−/−) in FIG. 4.). The complex biophysical model, however, takes into account the combined influence of these two parameters on the oral administration. Accordingly, under particular circumstances (sufficient solubility), even a substance with a molecular weight >500 and a lipophilicity >5 is capable of permeating the intestinal membrane and therefore being orally absorbed. Examples of such substances, which can be passively absorbed well in spite of high lipophilicity and high molecular weight, are itraconazoles (De Beule K., Van Gestel J., Drugs. 2001; 61 Suppl. 1: pp. 27-37), paclitaxel [Walle and Walle, Drug Metab Dispos. 1998 Apr. 26(4): 343-6] or cyclosporins [Fricker et al., Br. J. Pharmacol. 1996 Aug. 0.118 (7): 1841-7]. Conversely, there are known passively absorbed substances with a low lipophilicity and a low molecular weight which may be expected to have good absorption according to the Lipinski rules (i.e. they are inter alia layer in the region (+/+) in FIG. 4), but which are nevertheless orally absorbed only weakly. One such example is the substance ganciclovir [Wessel et al., J. Chem. Inf. Comput. Sci. 1998, 38, 726-735]. The biophysical model, however, correctly predicts a fraction dose absorbed of less than 8% for this substance. These examples illustrate the superiority of the biophysical model approach over the statistical selection criteria for simple physicochemical parameters.

The second example shows a selection of ADME maps for a data record of commercially available substances with various indication fields. The following measurement values were experimentally collected for the substances contained in this data record: membrane affinity as a measure of the lipophilicity (LogMA), binding constant to human serum albumin (LogHSA), both based on the TRANSIL® technology developed by Nimbus, Leipzig. The effective molecular weight (MW) is obtained simply from the respective empirical formula of the substance. The waters solubilities and the typical administered dosages of these commercially available products are furthermore known from the literature.

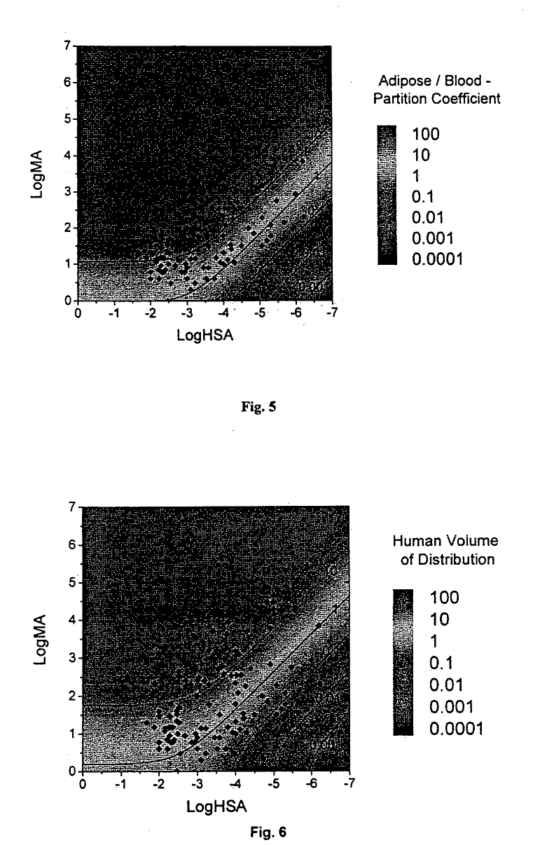

The ADME maps in FIGS. 5 to 10 show by way of example a selection of commercially available pharmaceutical substances. The substance names and the associated experimental measurement values for their physicochemical properties are summarised in Table 1.

The data points in the ADME maps of FIG. 11 represent a selection of agrochemical active agents, the relevant physicochemical parameters of which are listed in Table 2.

The organ-blood distribution coefficients for the various organs in FIGS. 5 to 8 were found according to the method described in DE0010160270 (page 5 starting at paragraph [0051] by using the data in FIG. 3).

FIG. 5 shows by way of example the map for the fat/plasma distribution coefficient, which was found according to the method described in DE 101 60 270 A1.

FIG. 6 shows by way of example the map for the human distribution volume, which was found according to the method described in DE 101 60 270 A1.

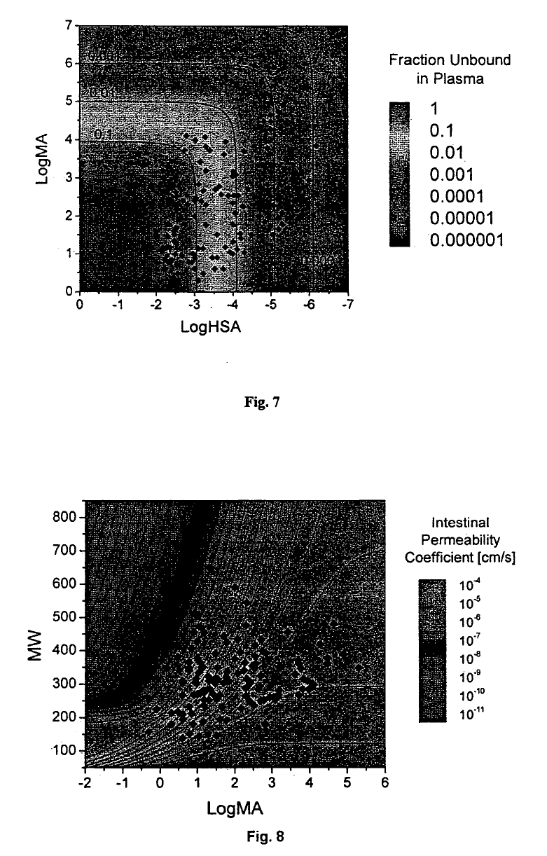

FIG. 7 shows by way of example the map for the fraction unbound in plasma, which was found according to the method described in DE 101 60 270 A1.

FIG. 8 shows by way of example the map for the intestinal permeability coefficient, which was found according to the method described in DE 101 60 270 A1.

FIG. 9 shows by way of example the map for the maximum absorbed dose in humans in the permeation-limited case, which was found according to the described method with the aid of a physiology-based pharmacokinetic model.

FIG. 10 shows by way of example the map for the absorbed dose in humans in the permeation- or solubility-limited case, which was found according to the method described in DE 101 60 270 A1 with the aid of a physiology-based pharmacokinetic model.

The ADME map for the phloem mobility in FIG. 11 was found with a PBPK model for plants, which is fully described in Satchivi et al. (Satchivi N. M., Stoller, E. W., Wax L. M., Briskin D. P., A nonlinear dynamic simulation model for xenobiotic transport and whole plant allocation following foliar application Parts I and II. Pest. Biochem. and Physiol. 2000; 68: 67-95).

Such ADME maps can be used particularly well in a research project, in order to obtain an intuitive graphical overview of the ADME properties of a library of substances. The ranking is carried out in combination with indication-specific rules. Such indication-specific rules may, for example, define a threshold value for the fraction unbound in plasma, a limit value for the fat/plasma distribution coefficient, a threshold value for the distribution volume or the fraction of the orally absorbed dose. In the physicochemical parameter space, such limit values for ADME properties represent nonlinearly bounded regions which result from the underlying biophysical models (see FIGS. 5-10). The preferential region may be highlighted in colour (for example by modulating the colour saturation). Substances which fulfil the requirement profile may then easily be selected and highlighted. When a plurality of such preferential regions are combined by means of Boolean algebra, a classification of the substances may be made in relation to the preferred ADME profile. Further information may be visualised by colour and/or size modulation of the data points.

The use of the described technique is not restricted to applications in the field of pharmaceutical research, from which the examples described above come. Utilisation is also possible in other fields for which ADME properties of substances are important, and where biophysical models are available for calculating them. One example is the distribution of crop protection agents or other substances in plants. Owing to the large pH differences inside the plant, the transport in the plant depends not only on the lipophilicity of the substances but also strongly on their pKa values. One important property is the distribution of substances from treated leaves into other parts of the plant (the so-called phloem mobility). FIG. 11 shows a corresponding contour-encoded property map, in which regions of strong translocation (contour values >10⁻¹) and weak translocation (contour values <10⁻³) can be seen. These property maps were set up by means of the described physiology-based plant model. It can be seen clearly that, here again, classification of the indicated data points is not possible with simple rules, which rely on the values of lipophilicity and pKa, whereas substances with a particular distribution behaviour can be readily identified according to the method described above.

TABLE 1
Compound list and parameters

| | | | | Solubility | Typical Mass or | | |
|---|---|---|---|---|---|---|---|
| # | COMPOUND | MW | LogMA | [mg/L] | Dose (p.o.) | | LogHSA |
| 1 | Acebutolol | 336 | 1.792 | 259 | 300 | mg | −2.301 |
| 2 | Acetylsalicylic acid | 180 | 0.301 | 4600 | 1200 | mg | −3.097 |
| 3 | Acyclovir | 225 | −0.097 | 33990 | 100-600 | mg | −2.000 |
| 4 | Alprenolol | 249 | 2.699 | 547 | 100 | mg | — |
| 5 | Amiloride | 214 | 0.301 | 1256 | 20 | mg | — |
| 6 | Amlodipine | 377 | 4.477 | 60 | 5-10 | mg | −5.000 |
| 7 | Amoxicilin | 365 | 0.778 | 3433 | 375-1000 | mg | −2.301 |
| 8 | Ampicillin | 349 | 1.114 | 3574 | 500 | mg | −4.119 |
| 9 | Antipyrine | 188 | 1.146 | 23760 | 10 | mg/kg | −2.161 |
| 10 | Atenolol | 266 | 0.602 | 685 | 200 | mg | −2.000 |
| 11 | Betaxolol | 307 | 2.398 | 451 | 20 | mg | −3.187 |
| 12 | Bumetanide | 364 | 2.279 | 32 | 0.5 | mg | −5.076 |
| 13 | Captopril | 217 | 0.477 | 6857 | 100 | mg | −3.222 |
| 14 | Carbamazepine | 236 | 2.519 | 17.7 | 800-1200 | mg | −2.539 |
| 15 | Cefadroxil | 363 | 1.079 | 1110 | 500 | mg | — |
| 16 | Cephalexine | 347 | 0.778 | 1789 | 500 | mg | −2.357 |
| 17 | Cefazolin | 455 | 0.903 | 214 | — | | −3.456 |
| 18 | Cefmetazole | 472 | 1.079 | >500 | — | | — |
| 19 | Cefoperazone | 646 | 1.362 | 64.2 | — | | — |
| 20 | Cefoxitin | 427 | 1.000 | 105 | — | | — |
| 21 | Ceftazidime | 505 | 1.000 | >500 | — | | −2.495 |
| 22 | Ceftriaxone | 512 | 0.903 | 958 | 400 | mg | −3.585 |
| 23 | Cefuroxime | 381 | 0.477 | 145 | 500 | mg | −2.553 |
| 24 | Chloramphenicol | 287 | 2.301 | 389 | 250 | mg | −2.824 |
| 25 | Chlorpromazine | 301 | 4.075 | 2.55 | 50-100 | mg | −3.284 |
| 26 | Cimetidine | 252 | 1.176 | 10460 | 200 | mg | −1.876 |
| 27 | Ciprofloxacin | 315 | 0.954 | 11480 | 200 | mg | −2.675 |
| 28 | Clomipramin | 297 | 4.104 | 0.294 | 50 | mg | −2.796 |
| 29 | Clonidine | 194 | 1.602 | 13580 | 0.3 | mg | −2.463 |
| 30 | Clozapine | 309 | 3.951 | 11.8 | 300-600 | mg | −3.814 |
| 31 | Caffeine | 194 | 0.602 | 2632 | 1-300 | mg | −2.222 |
| 32 | Corticosterone | 346 | 2.531 | 143 | 1-5 | mg | −3.886 |
| 33 | Coumarin | 146 | 1.505 | 1900 | — | | −3.584 |
| 34 | Despiramine | 266 | 3.725 | 0.99 | 50 | mg | −3.398 |
| 35 | Dexamethasone | 392 | 2.833 | 93 | 1.5 | mg | −3.114 |
| 36 | Diazepam | 267 | 3.170 | 59 | 10-20 | mg | −4.010 |
| 37 | Diclofenac | 260 | 2.940 | 5.61 | 50 | mg | −6.000 |
| 38 | Dicloxacillin | 434 | 2.079 | 3.63 | 250-500 | mg | −4.064 |
| 39 | Digoxin | 391 | 1.477 | 64.8 | 1.2 | mg | −2.284 |
| 40 | Diltiazem | 415 | 2.544 | >1000 | 180 | mg | −4.031 |
| 41 | Doxorubicin | 544 | 2.322 | 93 | 50-60 | mg | −1.886 |
| 42 | Enalapril | 376 | 1.000 | 35 | 10 | mg | −2.959 |
| 43 | Enalaprilate | 348 | 0.176 | 11 | 10 | mg | — |
| 44 | Enoxacin | 304 | 0.954 | 34300 | 200 | mg | −2.278 |
| 45 | Etoposide | 589 | 2.000 | 59 | 10-600 | mg | −4.699 |
| 46 | Felodipine | 348 | 5.301 | 20 | 27.5 | mg | −5.301 |
| 47 | Fleroxacin | 321 | 1.301 | 7320 | 400 | mg | −2.523 |
| 48 | Fluconazole | 274 | 1.176 | 1086 | 50-150 | mg | −2.357 |
| 49 | Flunitrazepam | 354 | 2.477 | 72.8 | 1 | mg | −3.046 |
| 50 | Fluoxetin | 261 | 3.049 | 2500 | 30 | mg | −4.000 |
| 51 | Fluvastatin | 395 | 2.146 | 0.47 | 2-10 | mg | −5.678 |
| 52 | Furosemide | 315 | 1.342 | 149 | 40 | mg | −2.469 |
| 53 | Ganciclovir | 255 | −0.301 | 28340 | 50-100 | mg | −1.000 |
| 54 | Gentamicin | 478 | 0.000 | 1000000 | — | | −2.469 |
| 55 | Glibenclamid | 476 | 2.301 | 35 | 1.25-5 | mg | −6.221 |
| 56 | Guanabenz | 209 | 0.602 | 1055 | 16-32 | mg | −3.745 |
| 57 | Haloperidol | 360 | 3.723 | 14 | 20 | mg | −2.745 |
| 58 | Hydrochlorothiazide | 280 | 1.146 | 1292 | 12.5-75 | mg | −3.222 |
| 59 | Hydrocortisone | 433 | 2.716 | 219 | 200 | mg | −4.398 |
| 60 | Ibuprofen | 192 | 1.778 | 2440 | 400 | mg | −5.301 |
| 61 | Imipramine | 280 | 3.190 | 1 | 40-60 | mg | −3.301 |
| 62 | Indomethacin | 340 | 2.255 | 3.11 | 50 | mg | −4.260 |
| 63 | Isradipin | 371 | 3.699 | 49 | 5-20 | mg | −4.301 |
| 64 | Ketoprofen | 254 | 1.505 | 120 | 25-200 | mg | −5.056 |
| 65 | Labetalol | 328 | 3.620 | 73 | 600 | mg | −3.770 |
| 66 | Lidocaine | 319 | 1.771 | 4100 | — | | −3.301 |
| 67 | Lisinopril | 406 | 0.845 | 13 | 10-20 | mg | −2.215 |
| 68 | Methylprednisolone | 374 | 2.000 | 123 | 0.6 | mg/kg | −3.000 |
| 69 | Metolazone | 348 | 1.531 | 133 | 2.5 | mg | −4.201 |
| 70 | Metoprolol | 267 | 1.591 | 4777 | 300 | mg | −2.000 |
| 71 | Metotrexate | 454 | 1.176 | 26000 | 0.1-10 | mg | −3.796 |
| 72 | N-Acetylprocainamide | 277 | 1.176 | >1000 | 0.5-2.5 | mg | −2.268 |
| 73 | Nadolol | 309 | 0.602 | 22400 | 80 | mg | — |
| 74 | Naproxen | 230 | 1.653 | 145 | 250 | mg | −5.208 |
| 75 | Nicardipine | 480 | 4.646 | | 20-30 | mg | −5.000 |
| 76 | Nifedipine | 346 | 3.778 | 56.3 | 30-60 | mg | −4.912 |
| 77 | Nimodipine | 419 | 4.279 | 24.3 | 30 | mg | −5.000 |
| 78 | Nisoldipine | 388 | 4.903 | 25 | 10-20 | mg | −5.319 |
| 79 | Nitrendipine | 360 | 4.477 | 77 | 20 | mg | −4.824 |
| 80 | Nordiazepam | 253 | 3.086 | 57 | 10 | mg | −4.097 |
| 81 | Norfloxacin | 303 | 0.602 | 177900 | 400 | mg | −3.000 |
| 82 | Ondansetron | 293 | 2.672 | 5.7 | 8 | mg | −3.237 |
| 83 | Oxacepam | 313 | 2.342 | 179 | 15 | mg | −4.000 |
| 84 | Oxprenolol | 265 | 2.301 | 3182 | 160 | mg | −3.301 |
| 85 | Paracetamol | 151 | 0.778 | 30350 | 500-1000 | mg | −2.000 |
| 86 | Pefloxacin | 315 | 1.519 | 11400 | 400-600 | mg | −2.469 |
| 87 | Penicilin G | 334 | 1.041 | 50 | 500 | mg | −4.301 |
| 88 | Pentazocin | 285 | 2.633 | | 50 | mg | −2.510 |
| 89 | Pentobarbital | 226 | 2.176 | 679 | 50-100 | mg | −2.523 |
| 90 | Phenobarbital | 232 | 0.778 | 1110 | 30-200 | mg | −2.854 |
| 91 | Phenoxymethyl penicillinic acid | 350 | 1.146 | 101 | 22 | mg | −3.102 |
| 92 | Phenylbutazone | 308 | 1.845 | 47.5 | 300 | mg | −4.839 |
| 93 | Phenytoin | 252 | 2.778 | 1267 | 400 | mg | −3.639 |
| 94 | Pindolol | 248 | 2.398 | 7883 | 15 | mg/kg | −2.699 |
| 95 | Piroxicam | 331 | 2.079 | 521 | 20 | mg | −5.301 |
| 96 | Practolol | 252 | 1.301 | 4472 | 25-600 | mg | — |
| 97 | Prazosin | 383 | 2.544 | 310 | 1 | mg | −4.141 |
| 98 | Prednisolone | 360 | 2.568 | 221 | 10-50 | mg | −3.694 |
| 99 | Probenecid | 285 | 1.322 | 27.1 | 500 | mg | −4.004 |
| 100 | Procainamide | 235 | 1.176 | 9450 | 4.5 | mg | −2.301 |
| 101 | Progesteron | 314 | 3.843 | 5 | 1-2.5 | mg | −6.189 |
| 102 | Promazine | 284 | 3.823 | 14.2 | 100 | mg | −3.301 |
| 103 | Promethazine | 284 | 3.992 | 15.6 | 25-200 | mg | −3.028 |
| 104 | Propranolol | 259 | 3.146 | 609 | 300 | mg | −4.028 |
| 105 | Quinidine | 324 | 2.699 | 104 | 330 | mg | −3.539 |
| 106 | Ranitidine | 272 | 1.079 | 24660 | 40-80 | mg | −2.155 |
| 107 | Salicylic acid | 138 | 0.602 | 3808 | 100-500 | mg | −3.523 |
| 108 | Scopolamine | 303 | 1.462 | 17400 | — | | −2.301 |
| 109 | Sotalol | 309 | 1.301 | 136800 | 240 | mg | −1.678 |
| 110 | Sulfamethizole | 270 | 1.000 | 1050 | 500-1000 | mg | −4.000 |
| 111 | Sulfasalacine | 398 | 2.748 | 2.44 | 2000 | mg | −5.469 |
| 112 | Sulpiride | 341 | 0.778 | 2275 | 200 | mg | −2.933 |
| 113 | Tenidap | 303 | 1.699 | 2676 | 120 | mg | — |
| 114 | Terazosin | 424 | 1.785 | 205 | 7.5 | mg | −4.000 |
| 115 | Terbutaline | 211 | 0.845 | 212800 | 5 | mg | −2.398 |
| 116 | Testosteron | 180 | 2.826 | 68 | 20 | mg | −4.912 |
| 117 | Tetracycline | 444 | 1.699 | 231 | 500-1000 | mg | −2.523 |
| 118 | Theophiline | 180 | 0.886 | 2800 | 400-800 | mg | — |
| 119 | Timolol | 316 | 1.477 | 2741 | 30 | mg | −2.000 |
| 120 | Tolbutamid | 270 | 1.415 | 109 | 1000 | mg | −4.211 |
| 121 | Tranexamic acid | 157 | −0.301 | 25000 | 500-2000 | mg | −4.000 |
| 122 | Trihexylphenidyl | 302 | 2.415 | 10000 | 1-4 | mg | −3.006 |
| 123 | Valproic acid | 144 | 1.041 | 895 | 600 | mg | −3.921 |
| 124 | Verapamil | 455 | 3.425 | 4.47 | 80-160 | mg | −3.155 |
| 125 | Warfarin | 308 | 1.505 | 17 | 5 | mg | −4.492 |
| 126 | Zidovudine | 267 | 0.903 | 311 | 10 | mg/kg | −3.000 |

TABLE 2
Crop Protection Example

| Name | logP | pKa |
|---|---|---|
| metsulfuron | 2 | 3.3 |
| metsulfuron-methyl | 1.2 | 3.3 |
| halosulfuron | −0.0186 | 3.44 |
| halosulfuron-methyl | 2.9 | 3.44 |
| primisulfuron | 0.06 | 3.47 |
| amidosulfuron | 1.63 | 3.58 |
| azimsulfuron | 2.1 | 3.6 |
| chlorsulfuron | 1.9 | 3.6 |
| prosulfuron | 2.8 | 3.76 |
| imazosulfuron | 2.7 | 4 |
| rimsulfuron | 1.3 | 4 |
| thifensulfuron | 0.02 | 4 |
| thifensulfuron-methyl | 1.23 | 4 |
| chlorimuron | 0.11 | 4.2 |
| chlorimuron-ethyl | 2.7 | 4.2 |
| triflusulfuron-methyl | 3.4 | 4.4 |
| nicosulfuron | 0.5 | 4.6 |
| triasulfuron | 1.6 | 4.64 |
| flupyrsulfuron-methyl- | 1.3 | 4.9 |
| sodium | | |
| tribenuron | −0.44 | 5 |
| tribenuron-methyl | 1.5 | 5 |
| oxasulfuron | 1.1 | 5.1 |
| bensulfuron | 0.62 | 5.2 |
| bensulfuron-methyl | 2.45 | 5.2 |
| sulfometuron | −0.51 | 5.2 |
| sulfometuron-methyl | 1.4 | 5.2 |
| diclofop | 4.5 | 3.43 |
| 2,4-D | 2.58 | 2.73 |
| MCPA | 2.75 | 3.07 |
| dichlorprop-P | 2.58 | 3.67 |
| dicamba | 2.8 | 1.97 |
| bifenox | 4.5 | 13.8 |
| quinclorac | 3.6 | 4.34 |
| metosulam | 2.5 | 4.8 |
| bromacil | 1.88 | 9.27 |
| diuron | 2.85 | 13.8 |
| monolinuron | 2.2 | 13.8 |

The foregoing is only a description of a non-limiting number of embodiments of the present invention. It is intended that the scope of the present invention extend to the full scope of the appended issued claims and their equivalents.

1. A method of selecting one or more compounds having a desirable profile of ADME properties in a biophysical model, the method comprising:
a) determining and/or selecting molecular properties of one or more chemical compounds in a computer system,
b) preparing one or more ADME maps, wherein each ADME map comprises data points representing the one or more compound's map position based on determining the one or more compound's combined values of desired properties in a suitable biophysical model and wherein the one or more compounds are within a selected molecular weight range,
c) linking the one or more compounds in a) with the biophysical model in b) and optionally representing the one or more compounds as data points in the ADME maps,
d) defining an indication-specific target profile of preferred ADME properties to be possessed by the one or more compounds, and
e) grouping the one or more compounds with respect to the indication-specific target profile in order to provide a classification of the compounds, and selecting one or more desired compounds with the aid of the classification.

2. The method according to claim 1, wherein the molecular properties are selected from the group consisting of lipophilicity, binding constant to plasma proteins, molecular weight, molecular volume, water solubility, solubility in intestinal fluid, permeability coefficient across a biological membrane, fraction unbound in plasma, kinetic constants of a metabolism process, and kinetic constants of an active transport process.

3. The method according to claim 1, wherein the biophysical model is selected from the group consisting of a physiology-based pharmacokinetic model for mammals, a physiology-based pharmacokinetic model for insects, and a physiology-based pharmacokinetic model for plants.

4. The method according to claim 1, wherein the ADME properties are selected from the group of properties related to a physiology-based pharmacokinetic model for mammals, the group consisting of the unbound fraction in plasma, organ/blood distribution coefficient, organ/plasma distribution coefficient, distribution volume, terminal half-life in blood, terminal half-life in plasma, terminal half-life in an organ, intestinal permeability, fraction of a dose of the substance absorbed following oral application, and the maximum concentration in the blood, plasma or an organ.

5. The method according to claim 1, wherein the target profile is obtained from empirical values, expert knowledge and/or the statistical distribution of relevant ADME properties for known compounds.

6. The method according to claim 1, wherein the classification is carried out using truth values which represent a fulfilment of an individual requirement of an ADME property.

7. The method according to claim 1, wherein the classification is performed by combining a plurality of truth values, which represent a fulfilment of an individual requirement, by means of Boolean algebra.

8. The method according to claim 1, wherein the classification is performed by means of an index value, which quantifies a deviation from a target value.

9. The method according to claim 1, wherein the classification is performed by means of a weighted average of a plurality of index values, which quantify a deviation from a target value.

10. The method according to claim 1, wherein the classification is performed by means of a probability value, which indicates a probability rank in relation to an empirical distribution function obtained from known substances for an ADME property.

11. The method according to claim 1, wherein the ADME properties are selected from a group of properties related to a physiology-based pharmacokinetic model for plants, said group of properties consisting of the rate of absorption into a leaf following a spray application, the rate of phloem mobility, and the rate of xylem mobility.

12. The method according to claim 1, wherein the ADME properties are selected from a group of properties related to a physiology-based pharmacokinetic model for insects, said group of properties consisting of the rate of absorption in an insect gut, and the rate of absorption through an insect cuticle.
